(19) United States
(12) Patent Application Publication
(10) Pub. No.: US 2009/0215071 A1
(43) Pub. Date: Aug. 27, 2009

(54) METHODS OF TARGETING BAFF

(76) Inventors: Teresa Cachero, Quincy, MA (US); Adrian Whitty, Hopkinton, MA (US); Alexey Lugovskoy, Woburn, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/996,832

(22) PCT Filed: Jul. 27, 2006
(86) PCT No.: PCT/US06/29767

§ 371 Date: Jun. 23, 2008

Related U.S. Application Data
(60) Provisional application No. 60/703,190, filed on Jul. 28, 2005.

(51) Int. Cl. G01N 33/53; C07K 16/18; C07K 14/525

(52) U.S. Cl. 435/7.1; 530/389.1; 530/395

(57) ABSTRACT

The present disclosure provides compositions and methods relating to the structure of BAFF in solution. The disclosure includes BAFF 60-mers, BAFF trimers, methods of making BAFF 60-mers and BAFF trimers, antibodies that preferentially bind one form or the other, and methods of identifying or evaluating a compound on the basis of its relative binding to or activity towards a BAFF 60-mer and a BAFF trimer. The disclosure also provides computer-based systems and methods relating to BAFF structures.

This application claims priority to U.S. Provisional Application No. 60/703,190, filed Jul. 28, 2005, the contents of which are incorporated herein by reference.

### FIELD OF THE INVENTION

The present invention relates to BAFF, a B-cell activating factor in the TNF family. The invention further relates to the structure of BAFF in solution and to methods and compositions relating to the structure of BAFF.

### BACKGROUND

BAFF (B cell-activating factor), also known as BLyS, TALL-1, THANK and zTNF4, is a member of the TNF family that is expressed in macrophages, monocytes, dendritic cells and T cells and is critical for the survival of B cells. Moore et al., Science 285:260-263 (1999); Mukhopadhyay et al., J. Biol. Chem. 274:15978-15981 (1999); Gross et al., Nature 404:995-999 (2000); Shu et al., J. Leukoc. Biol. 65:680-683 (1999). BAFF is a type 11 transmembrane protein that can be proteolytically cleaved between Arg 133 and Ala 134 and released as a soluble protein. Moore et al., Science 285:260-263 (1999); Schneider et al., J. Exp. Med. 189:1747-1756 (1999). The solution structure of BAFF at physiological pH has been a matter of debate.

### SUMMARY

The present invention is based, in part, on the discovery that both trimers and higher order oligomers (e.g., 60-mers) of BAFF are biologically active and have different biological activity. Accordingly, compositions and methods relating to these distinct BAFF structures are described herein. In part, the present disclosure provides methods and compounds that distinguish or differentiate between the BAFF 60-mer and the BAFF trimer. Such methods are useful, e.g., to provide and/or modulate BAFF preparations having different activity, e.g., different levels of activity in a B cell assay disclosed herein.

In one aspect, the disclosure provides a method of identifying a compound that binds (and optionally either inhibits or agonizes) one BAFF structure with a higher affinity than another BAFF structure, i.e., a compound that preferentially binds either a 60-mer or a trimer, relative to each other. In one aspect, the method includes the steps of providing a test compound, allowing the test compound to interact with a BAFF trimer and/or a 60-mer, determining whether the test compound preferentially binds the trimer or the 60-mer, and selecting a test compound that preferentially binds either the 60-mer or the trimer, thereby identifying a BAFF binding compound with preferential binding affinity to a BAFF trimer or 60-mer. In some embodiments, the test compound is an element of a library, e.g., a phage display library or other peptide or antibody library, a small molecule library, or aptamer library. In some embodiments, the test compound and selected compound thus identified is an antibody, a peptide, an aptamer or a small molecule. The identified compound is optionally further evaluated for its effect on BAFF activity, e.g., its ability to inhibit a BAFF-related activity in vitro or in vivo, e.g., its ability to inhibit BAFF receptor binding, B cell survival or proliferation, Ig secretion, or activity in an animal model of disease (e.g., a model of autoimmune disease such as rheumatoid arthritis, lupus, multiple sclerosis, psoriasis, or Crohn's Disease). In certain embodiments, the BAFF trimer of the method includes a mutation of at least one amino acid in the DE loop. In a further embodiment, the trimer includes at least one monomeric subunit having one, two or three of the following mutations: substitution of Lys 216 with aspartate (Asp) or glutamate (Glu); substitution of His 218 with glycine (Gly), alanine (Ala), or serine (Ser); and substitution of Glu 223 with arginine (Arg) or lysine (Lys). In further embodiments, the trimer includes at least one His218Ala mutation.

The disclosure also provides isolated antibodies that bind one BAFF structure with a higher affinity than another BAFF structure, i.e., antibodies that preferentially bind either a trimer or a higher order oligomer, such as a 60-mer, relative to each other. In some embodiments, the antibody preferentially binds BAFF 60-mer; in other embodiments, the antibody preferentially binds the BAFF trimer. In further embodiments, the binding constants for the antibody and the trimer and the antibody and the 60-mer, respectively, differ by at least a factor of 5, 10, 20, 30, 40, 50, 100, 200, 500, 1000, or more. In part, these antibodies include an antibody identified by any of the methods described herein. The disclosure also provides pharmaceutical compositions that include any of the aforementioned antibodies.

The disclosure also provides BAFF trimers and BAFF 60-mers, and methods of making the same. In one embodiment, the disclosure provides a BAFF trimer comprising a mutation in the DE loop. In a further embodiment, the mutation is a deletion of at least one of Lys 216 and/or His 218; i.e., at least one monomeric subunit in the trimer comprises a deletion of Lys 216 and/or His 218. In one embodiment, the trimer includes at least one monomeric subunit having one, two or three of the following mutations: substitution of Lys 216 with aspartate (Asp) or glutamic acid (Glu); substitution of His 218 with glycine (Gly), alanine (Ala), or serine (Ser); and substitution of Glu 223 with arginine (Arg) or lysine (Lys). In a further embodiment, at least one monomeric subunit in the trimer comprises a His218Ala mutation. In one embodiment, the BAFF trimer is active. In one aspect, BAFF activity may be determined by testing for binding to a BAFF receptor. In one aspect, BAFF activity may be determined by assaying for biological activity as described herein.

The disclosure also provides a method of making a BAFF trimer, comprising constructing or preparing a BAFF polypeptide having at least one substitution or deletion at His 218, Lys 216 or Glu 223. In one embodiment, the method includes constructing or preparing a BAFF polypeptide having at least one mutation selected from the following: substitution of Lys 216 with a natural or non-natural amino acid that has full or partial negative charge on any of its sidechain atoms or an organic moiety that has full or partial negative charge on any of its atoms; substitution of His 218 with a natural or non-natural amino acid or organic moiety that has a molecular weight of 114 Da or lower; and substitution of Glu 223 with a natural or non-natural amino acid that possesses full or partial positive charge on any of its sidechain atoms or any organic moiety that has full or partial positive charge on any of its atoms. For example, the method can include substitution of Lys 216 with aspartate (Asp) or glutamic acid (Glu); substitution of His 218 with glycine (Gly), alanine (Ala), or serine (Ser); and substitution of Glu 223 with arginine (Arg) or lysine (Lys). In one embodiment, the BAFF trimer is biologically active.

The disclosure also provides another method of making a BAFF trimer. The method includes constructing or preparing a soluble BAFF polypeptide that has an extended or modified N-terminus, e.g., constructing or preparing a soluble BAFF polypeptide that has one or more (e.g., 2 or 3) of the following characteristics in its N-terminus: (a) it has an N-terminal amino acid selected from amino acids 84-141 of a BAFF polypeptide (e.g., amino acids 84-141 of a human BAFF polypeptide, e.g., amino acids 84-141 of SEQ ID NO:1 or a functional variant thereof); (b) it has an N-terminal chemical modification, e.g., it is PEGylated at its N-terminus; (c) it comprises a heterologous amino acid sequence at its N-terminus, e.g., it comprises an N-terminal tag of at least 7 amino acids (e.g., between 7 and 100 amino acids, between 15 and 80 amino acids, between 20 and 50 amino acids) or it is fused at its N-terminus (with or without a spacer) to a second polypeptide, such as an Fc fragment of an Ig molecule; and (d) it has an additional amino acid sequence at its N-terminus that contains one or more glycosylation sites leading to the incorporation of one or more glycans at or near the N-terminus during expression.

The disclosure also provides BAFF 60-mers and methods of making the same. In one embodiment, the disclosure provides a BAFF 60-mer having at least one mutation in amino acids 134-216 or amino acids 225-285 and having the native sequence of BAFF, or conservative substitutions thereof, in amino acids 217 to 224. In a further embodiment, this BAFF 60-mer includes at least one deletion in amino acids 134 to 145. In a further embodiment, amino acids 1 to 145 are deleted. In one embodiment, the disclosure provides a BAFF 60-mer wherein at least one monomeric subunit comprises a substitution of His 218 with an amino acid selected from the group consisting of Trp, Phe, Tyr, Met, Ile, and Leu.

The disclosure also provides a method of making a BAFF 60-mer, comprising constructing or preparing a BAFF polypeptide having at least one substitution or deletion at His 218, Lys 216, or Glu223. In one embodiment, the method includes constricting or preparing a BAFF polypeptide having at least one mutation selected from the following: substitution of His 218 with a natural or non-natural amino acid or organic moiety that has a molecular weight of 115 Da or higher; substitution of Lys 216 with a non-polar or uncharged aromatic natural or non-natural amino acid or an organic moiety that will have similar properties, combined with substitution of Glu 223 with a non-polar or uncharged aromatic natural or non-natural amino acid or an organic moiety with the same properties. In one embodiment, the method includes constructing or preparing a BAFF polypeptide having a substitution of His 218 with an amino acid selected from the group consisting of Trp, Phe, Tyr, Met, Ile, and Leu. In one embodiment, the BAFF 60-mer is biologically active.

The disclosure also provides a method of evaluating a BAFF-binding compound, comprising providing the compound, allowing it to interact with a BAFF trimer and/or a BAFF 60-mer, determining the activity of the compound toward the BAFF trimer and the BAFF 60-mer, and thereby evaluating the activity of the compound, e.g., determining whether the compound preferentially binds, inhibits and/or agonizes the trimer or 60-mer. In some embodiments, the compound thus evaluated is an antibody, a peptide, an aptamer, or a small molecule. In certain embodiments, the BAFF trimer of the method includes a mutation of at least one amino acid in the DE loop. In a further embodiment, the trimer includes at least one monomeric subunit having one of the following mutations: substitution of Lys 216 with aspartate (Asp) or glutamate (Glu); substitution of His 218 with glycine (Gly), alanine (Ala), or serine (Ser); and substitution of Glu 223 with arginine (Arg) or lysine (Lys). In further embodiments, the trimer includes at least one His218Ala mutation.

The disclosure also provides computational methods of designing, analyzing, or identifying BAFF 60-mers, BAFF trimers, BAFF-binding compounds, BAFF agonists, and BAFF antagonists.

### BRIEF DESCRIPTION OF THE DRAWINGS

FIG. 1 shows size-exclusion chromatography data (in 10 mM Tris pH 7.5, 150 mM NaCl) obtained using Superdex 200 10/30. Absorbance at 280 nm of molecular weight standards is represented in grey: A=Thyroglobulin (670 kDa), B=Gamma globulin (158 kDa), C=Ovalbumin (44 kDa) and D=Myoglobulin (17 kDa). 245 pM (line 1), 1.22 nM (line 2), 2.45 nM (line 3), 4.90 nM (line 4), 19.6 nM (line 5) and 98 nM (line 6) (molarities calculated from BAFF 60mer) of A134-BAFF were loaded onto the gel filtration column and eluted with a flow rate of 0.5 ml/min and collected 500 μl per fraction. In all cases, A134-BAFF elutes as a high oligomer (>670 kDa). In the Y axes absorbance at 214 nm is represented. 20 μl of the appropriate fractions were assayed by Western blot using an antibody against the carboxy terminus of BAFF. 60-mer panel: lanes 1-6: 245 pM, 1.22 nM, 2.45 nM, 4.90 nM, 19.6 nM and 98 nM respectively of A134-BAFF eluted at the Mw corresponding to 60-mer. Trimer panel: lanes 1-6: 245 pM, 1.22 nM, 2.45 nM, 4.90 nM, 19.6 nM and 98 nM respectively of A134-BAFF eluted at the Mw corresponding to trimer.

FIG. 2 shows the results of analytical gel filtration experiments to determine the structure of various BAFF polypeptides. FIG. 2A shows the pH dependency of 60-mer formation. A134-BAFF-N242Q was analyzed under at pH 5.0 (dashed line) and pH 8.0 (solid line). Buffer conditions are described in Example 2. FIG. 2B shows that the H218A mutation abolishes 60-mer formation. A134-BAFF-N242Q (solid line) and A134-BAFF-H218A (dashed line) were analyzed in 10 mM Tris pH 7.5, 150 mM NaCl. FIG. 2C shows that myc-Q136-BAFF is trimeric even at high pH. myc-Q136-BAFF was characterized at pH 7.5 (dashed line) and 9.0 (solid line). The molecular weight markers are shown as in FIG. 1A (grey).

FIG. 3 shows the functional activity of BAFF 60-mer versus trimeric BAFF. FIG. 3A shows the proliferation of B cells induced by trimeric myc-Q136-BAFF (closed circles) versus trimeric A134-BAFF-H218A (closed squares) and 60-mers A134-BAFF-N242Q (open squares) and A134-BAFF (open circles). B cells were incubated in the presence of 5 ug/ml of F(ab′)2 fragment goat anti-mouse IgM antibody and with different concentrations of different forms of BAFF for 48 h. Cells were pulsed for an additional 18 hours with [3H]-thymidine (1 uCi/well) and harvested. [3H]-Thymidine incorporation was monitored by liquid scintillation counting. Each sample was analyzed in triplicate and error bars are represented. The data are representative of three independent experiments. FIG. 3B shows the affinity of myc-Q136-BAFF and A134-BAFF for monomeric BAFFR. The indicated concentrations of soluble, monomeric BAFFR were equilibrated in solution with a fixed concentration of BAFF (50 nM trimeric BAFF [myc-Q136-wt, closed circles] or 2.5 nM 60-mer BAFF [A134-BAFF-wt, open circles]). Solutions were then run over a BCMA-Fc derivitized surface as described in Example 3. The affinity of the solution phase binding of BAFFR with BAFF was determined by fitting the data to a quadratic binding equation as described. Day et al., Biochemistry 44:1919-1931 (2005).

FIG. 4 shows an alignment of the carboxy terminal portions of BAFF amino acid sequences from a variety of species (Homo sapiens, Mus musculus, Gallus gallus, Pan troglodytes, Tetraodon nigroviridis, Rattus norvegius, Canis familiaris, Bos taurus, and Pongo pygmaeus). For this figure only, the amino acids have been renumbered to correspond to the conserved carboxy-terminal domains. Due to divergent sequence lengths at the amino-termini, the corresponding positions in the full length sequences vary from species to species.

### BRIEF DESCRIPTION OF THE SEQUENCES

SEQ ID NO:1, SEQ ID NO:2, and SEQ ID NO:3 represent the full-length amino acid sequences of human, mouse, and chicken BAFF, respectively.

### DETAILED DESCRIPTION

This invention is based in part on the discovery that, under physiological conditions, BAFF can form trimers and higher order oligomers (e.g., 60-mers), both of which have distinct biological activity (e.g., have distinct effects on B cell proliferation). It has been found that 60-mer formation is not dependent upon the presence of an amino-terminal histidine tag and that a mutated BAFF that does not form 60-mers nonetheless retains activity. These findings reveal a need for BAFF 60-mers and BAFF trimers, and for methods of making the same. These findings also reveal a need for compounds (e.g., antibodies, peptides, aptamers, or small molecules) that bind preferentially to either a 60-mer or a trimer, and for methods of identifying such compounds. These experiments also reveal a need for compounds having different activity toward a BAFF 60-mer and a BAFF trimer, and for related methods of evaluating the activity of a compound. There is also a newfound need for computational methods relating to these BAFF structures.

### BAFF

BAFF knockout mice lack mature B cells in the periphery, showing that BAFF is required for B cell development in vivo. Gross et al., Immunity 15: 289-302 (2001); Schiemann et al., Science 293:2111-2114 (2001). Animals overexpressing BAFF display symptoms of autoimmune disorders (Mackay, J. Exp. Med. 190:1697-1710 (1999)) and soluble BAFF is detected in the blood of patients with various autoimmune disorders. Gross et al., Nature 404:995-999 (2000); Groom et al., J. Clin. Invest. 109:59-68 (2002); Zhang et al., J. Immunol. 166:6-10 (2001); Cheema et al., Arthritis Reum. 44:1313-1319 (2001). BAFF has also been reported to form biologically active heteromers with APRIL (a proliferation-inducing ligand), a related TNF family ligand. These heterotrimers are present in serum samples from patients with systemic immune-based rheumatic diseases. Roschke et al., J. Immunol. 169:4314-4321 (2002).

BAFF co-stimulates the proliferation of B cells in the presence of anti-IgM (Schneider et al., J. Exp. Med. 189:1747-1756 (1999)) and is able to signal through three receptors: B cell maturation antigen (BCMA), transmembrane activator and cyclophilin ligand interactor (TACI), and BAFF receptor (BAFFR, BR3). Fusion proteins of these receptors with the CH1, CH2, and hinge region of human IgG1 block the proliferation of B cells induced by BAFF. Gross et al., Nature 404:995-999 (2000); Gross et al., Immunity 15: 289-302 (2001); Thompson et al., J. Exp. Med. 192:129-135 (2000); Thompson et al., Science 293:2108-2111 (2001).

BCMA and TACI bind to APRIL as well as BAFF. Gross et al., Nature 404:995-999 (2000); Wu et al., J. Biol. Chem. 275:35478-35485 (2000); Xia et al., J. Exp. Med. 192:137-143 (2000); Yan et al., Nat. Immunol. 1:37-41 (2000); Yu et al., Nat. Immunol. 1:252-256 (2000). BAFFR is expressed in all peripheral B cells and is specific for BAFF, i.e., unlike BCMA and TACI, BAFFR does not bind APRIL. Mice lacking BAFFR have a similar phenotype to the BAFF knockout mice. Thompson et al., Science 293:2108-2111 (2001); Yan et al., Curr. Biol. 11:1547-1552 (2001). Recently, studies with monomeric receptors have shown that BAFF binds BAFFR with 100 fold higher affinity than it binds BCMA. Rennert et al., J. Exp. Med. 192:1677-1684 (2000); Patel et al., J. Biol. Chem. 279:16727-16735 (2004); Day et al., Biochemistry 44:1919-1931 (2005).

There is controversy in the field as to the structure of the biologically active form of BAFF. The first TNF family ligand to be structurally characterized was TNFα. The functional unit of TNFα is a trimer, with each monomer consisting entirely of P strands and loops. Jones et al., Nature 338:225-228 (1989); Eck and Sprang, J. Biol. Chem. 264:17595-17605 (1989). Subsequent studies revealed similar structures for TNFβ, CD40L, and TRAIL (Eck et al., J. Biol. Chem. 267:2119-2122 (1992); Karpusas et al., Structure 3:1031-1039 (1995); Cha et al., Immunity 11:253-261 (1999)), leading to speculation that all TNF family members might have similar structures, including trimeric functional units. Locksley et al., Cell 104:487-501 (2001); Fesik, Cell 103:273-282 (2000).

Initial studies of the structure of BAFF indicated that it shared the trimeric functional unit of other TNF family ligands. Karpusas et al. previously reported the crystal structure of the BAFF extracellular domain, using a construct starting at residue Gln 136 and with an amino terminal myc tag (myc-Q136-BAFF). Karpusas et al., J. Mol. Biol. 315:1145-1154 (2002). The structure, obtained at pH 4.5, showed two BAFF trimers per asymmetric unit in the crystal structure. Like other TNF family members, each monomer of BAFF was found to fold as a sandwich of two antiparallel α-sheets. The structure of BAFF revealed that the loop connecting β strands D and E is longer than the corresponding loops seen in other TNF family members. Karpusas et al., J. Mol. Biol. 315:1145-1154 (2002). Oren and coworkers independently reported the crystal structure of BAFF at pH 6.0, which also showed a dimer of trimers in the asymmetric unit. This structure includes a binding site for magnesium, which the authors suggest could be important for trimer stabilization. Oren et al., Nat. Struct. Biol. 9:288-292 (2002).

However, just prior to the publication of Oren et al., Liu et al. reported that a BAFF construct starting at residue Ala 134 and with an N-terminal histidine tag (His-A134-BAFF), displayed an oligomeric, virus-like structure containing 20 trimers (60 monomers, 60-mer) when crystallized at pH 9.0. Liu et al., Cell 108:383-394 (2002). Residues in the long DE loop appeared to contribute to stabilizing interactions in the trimer-trimer interface. Liu et al. also showed that formation of BAFF 60-mer in solution was pH dependent. Replacing eight residues in the DE loop with two glycine residues abolished 60-mer formation, resulting in a mixture of trimers and monomers. This mutant was inactive in both a transfected cell assay and a co-stimulation assay on B lymphocytes from healthy donors. Liu et al., Cell 108:383-394 (2002). The crystal structure of the BAFF:BAFFR and BAFF:BCMA complexes have also been solved, and shows a similar, virus-like BAFF 60-mer with each of the 60 receptor binding sites occupied by a BAFFR or a BCMA molecule. Liu et al., Nature 423:49-56 (2003).

More recently, however, a report by Zhukovsky and colleagues questioned the relevance of the BAFF 60-mer in solution. Zhukovsky et al., Nature 427:413-414 (2004). They suggested that BAFF, like other TNF family members, exists as a trimer and that the 60-mer formation reported by Liu et al. was an artefact of the histidine tag present in their construct. Furthermore, Zhukovsky and coworkers reported that histidine tagged and untagged BAFF protein showed equivalent activity in a cell-based assay.

In response, Liu et al. reiterated their position that the 60-mer is the biologically active form of BAFF. They also stated that, in their hands, size exclusion chromatography using an untagged version of soluble BAFF (amino acids 134 to 285) yielded results consistent with those expected of a 60-mer. Hong et al., Nature 427:414 (2004).

Thus, the field is divided between two contradictory positions, with each side insisting that only one BAFF structure is biologically relevant. Liu et al. maintain that the 60-mer is the physiologically relevant structure and is required for activity. In contrast, Zhukovsky et al. find that both forms are equally active, but contend that the trimer is the naturally occurring form of BAFF, with the 60-mer a mere artefact of the amino-terminal histidine tag.

The data described herein show that both the trimer and the 60mer have biological activity, and the biological activity of the trimer and 60-mer (e.g., activity in a B cell assay described herein) is distinct.

### BAFF Polypeptides

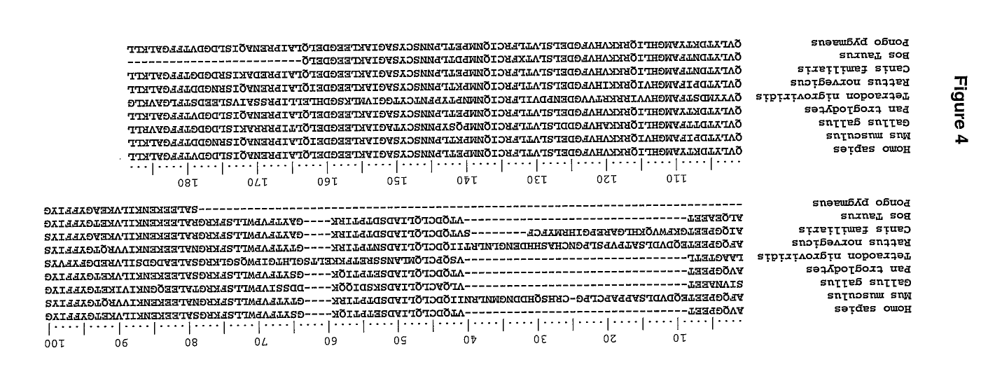

The amino acid and nucleic acid sequences of naturally occurring full-length human BAFF are available under GenBank™ accession Nos. AAD25356 (SEQ ID NO:1) and AF116456, respectively. The amino acid and nucleic acid sequences of full-length mouse BAFF are available under GenBank™ accession Nos. AAD22475 (SEQ ID NO:2) and AF119383, respectively. The amino acid and nucleic acid sequences of full-length chicken BAFF are available under GenBank™ accession Nos. AAP88060 (SEQ ID NO:3) and AY263378, respectively. An alignment of these sequences and the BAFF sequences from several other species is shown in FIG. 4.

Full-length BAFF is a type II membrane protein having intracellular, transmembrane, and extracellular domains. In human BAFF, these domains are comprised approximately (e.g., ±2 or 3 residues) of amino acids 1-46, 47-73, and 74-285 of SEQ ID NO:1, respectively. In mouse BAFF, these domains are comprised approximately (e.g., ±2 or 3 residues) of amino acids 1-53, 53-73, 74-309 of SEQ ID NO:2, respectively. A naturally occurring soluble form of BAFF exists, in which proteolytic cleavage occurs between amino acids R133 and A134 in human BAFF (amino acids R125 and A126 in mouse BAFF as predicted), resulting in a water-soluble biologically active C-terminal portion of BAFF.

Typically, a “BAFF polypeptide” is a polypeptide that includes a full length BAFF amino acid sequence (e.g., SEQ ID NO:1, 2 or 3) or a functional fragment or domain thereof (e.g., a soluble BAFF that includes all or part of the extracellular domain and excludes the transmembrane and intracellular domains; a soluble BAFF that includes at least the TNF-like domain and excludes the transmembrane and intracellular domains) and preferably has at least one BAFF biological activity. In some cases, a BAFF polypeptide can be a chimeric sequence comprising stretches of amino acids from BAFF sequences of different species. A BAFF polypeptide can also optionally include a heterologous (non-BAFF) amino acid sequence, wherein a BAFF polypeptide is fused to a heterologous amino acid sequence such as a peptide tag, AP, or an Fc region of an Ig, e.g., of an IgG. A human BAFF polypeptide can be a polypeptide at least 80%, 85%, 90%, preferably at least 95%, 96%, 98%, or 99% identical to SEQ ID NO:1 or to a soluble fragment of SEQ ID NO:1, having at least one BAFF biological activity, e.g., it binds BAFF-R, affects B cell proliferation, or has activity in any other BAFF functional assay described herein. Also included is a BAFF polypeptide that comprises SEQ ID NO:1 or a soluble fragment thereof as described herein with up to 15 amino acid deletions, substitutions, or additions, and has a functional activity of BAFF. Unless otherwise noted, descriptions of specific amino acid positions refer to the human sequence (SEQ ID NO:1) or homologous sequences in other BAFF homologues, as defined, e.g., by the alignment shown in FIG. 4. For example, a BAFF polypeptide, wherein His 218 is substituted with Ala, encompasses a sequence comprising the sequence of SEQ ID NO:2 with an alanine mutation at position 242, since this is the histidine residue of SEQ ID NO:2 that corresponds to His 218 of the human sequence.

BAFF polypeptides include soluble BAFF, whether naturally occurring or not. Such soluble forms of BAFF do not include the transmembrane and intracellular domains. Since naturally occurring soluble BAFF does not comprise a portion of the extracellular domain (i.e., amino acids 74-133 of SEQ ID NO:1 or amino acids 74-125 of SEQ ID NO:2), soluble BAFF of the invention may likewise exclude these regions. Alternatively, a soluble BAFF can include all or a portion of the extracellular domain larger than naturally-occurring soluble BAFF, e.g., a soluble BAFF may have an N-terminus at any of amino acids 74-145.

Within the extracellular domain, BAFF shares identity with other TNF family members: 28.7% with APRIL, 16.2% with TNF-α, and 14.1% with lymphotoxin (LT)-α. The extracellular domain of BAFF, including naturally occurring soluble BAFF, therefore contains the TNF-like domain delimited approximately (e.g., +2 or 3 residues) by amino acids 145-284 in SEQ ID NO:1 and amino acids 170-305 in SEQ ID NO:2. Accordingly, in certain embodiments, BAFF is a polypeptide comprising all or a substantial part of the TNF-like domain of BAFF, e.g., amino acids 145-284 of SEQ ID NO:1 (human BAFF), amino acids 170-305 of SEQ ID NO:2 (mouse BAFF), corresponding sequences indicated by the alignment presented in FIG. 4, chimeric sequences comprising amino acids from BAFF sequences of different species, fragments of any of the above, or sequences having at least 80%, 85%, 90%, or 95% sequence identity with any of the above. In one embodiment, a human BAFF polypeptide includes that TNF-like domain (amino acids 145-284 of SEQ ID NO:1) and has an N-terminal residue selected from amino acids 74-144 of SEQ ID NO:1.

Percent identity between two amino acid sequences may be determined by standard alignment algorithms such as, for example, Basic Local Alignment Tool (BLAST) described in Altschul et al. (1990) J. Mol. Biol., 215:403-410, the algorithm of Needleman et al. (1970) J. Mol. Biol., 48:444-453, or the algorithm of Meyers et al. (1988) Comput. Appl. Biosci., 4:11-17. Such algorithms are incorporated into the BLASTN, BLASTP, and “BLAST 2 Sequences” programs (see www.ncbi.nlm.nih.gov/BLAST). When utilizing such programs, the default parameters can be used. For example, for nucleotide sequences the following settings can be used for “BLAST 2 Sequences”: program BLASTN, reward for match 2, penalty for mismatch −2, open gap and extension gap penalties 5 and 2 respectively, gap x_dropoff 50, expect 10, word size 11, filter ON. For amino acid sequences, the following settings can be used for “BLAST 2 Sequences”: program BLASTP, matrix BLOSUM62, open gap and extension gap penalties 11 and 1 respectively, gap x_dropoff 50, expect 10, word size 3, filter ON.

In non-limiting illustrative embodiments, BAFF comprises amino acids 134-285 of SEQ ID NO:1, or N- and/or C-terminal truncations thereof. For example, the N-terminus of BAFF may be between residues 134-170 of SEQ ID NO:1, e.g., the N-terminus of BAFF may extend up to and include amino acid 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, or 170; while independently, the C-terminus be between residues 250-285 of SEQ ID NO:1, e.g., it may extend up to and include amino acid 285, 284, 283, 282, 281, 280, 279, 278, 277, 276, 275, 274, 273, 272, 271, 270, 269, 268, 267, 266, 265, 264, 263, 262, 261, 260, 259, 258, 257, 256, 255, 254, 253, 252, 251, 250 of SEQ ID NO:1. In one embodiment, BAFF comprises amino acids 136-285 of SEQ ID NO:1.

In other nonlimiting illustrative embodiments, BAFF comprises amino acids 126-309 of SEQ ID NO:2, or an N- and/or C-terminal truncations thereof. For example, the N-terminus of BAFF may extend up to and include amino acid 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, or 170; while independently, the C-terminus may extend to and include amino acid 309, 308, 307, 306, 305, 304, 303, 302, 301, 300, 299, 298, 297, 296, 295, 294, 293, 292, 291, 290, 289, 288, 287, 286, 285, 284, 283, 282, 281, 280, 279, 278, 277, 276, 275, 274, 273, 272, 271, or 270 of SEQ ID NO:2.

In one embodiment, a BAFF polypeptide of the invention is a naturally occurring variant of SEQ ID NO:1, SEQ ID NO:2, SEQ ID NO:3, or any of the related sequences shown in FIG. 4. BAFF polypeptides suitable for use in the methods of the invention further include derivatives of BAFF in which the native BAFF sequence is mutated, partially deleted, and/or contains one or more insertions so long as changes to the native sequence do not substantially affect the biological activity of the molecule. Such changes may involve, for example, conservative amino acid substitution(s) according to Table 1. Nonlimiting examples of such changes are shown in the BAFF sequences from various species aligned in FIG. 4. In certain embodiments, a BAFF polypeptide of the invention may contain no more than, for example, 50, 40, 30, 25, 20, 15, 10, 5, 4, 3, 2, or 1 amino acids that are substituted, deleted, or inserted relative to the naturally occurring BAFF sequences of SEQ ID NO:1, SEQ ID NO:2, or SEQ ID NO:3.

TABLE 1

| Original | Exemplary |
|---|---|
| Residues | Substitutions |
| Ala (A) | Val, Leu, Ile |
| Arg (R) | Lys, Gln, Asn |
| Asn (N) | Gln |
| Asp (D) | Glu |
| Cys (C) | Ser, Ala |
| Gln (Q) | Asn |
| Glu (E) | Asp |
| Gly (G) | Pro, Ala |
| His (H) | Asn, Gln, Lys, Arg |
| Ile (I) | Leu, Val, Met, Ala, Phe, Norleucine |
| Leu (L) | Norleucine, Ile, Val, Met, Ala, Phe |
| Lys (K) | Arg, 1,4-Diamino-butyric Acid, Gln, Asn |
| Met (M) | Leu, Phe, Ile |
| Phe (F) | Leu, Val, Ile, Ala, Tyr |
| Pro (P) | Ala |
| Ser (S) | Thr, Ala, Cys |
| Thr (T) | Ser |
| Trp (W) | Tyr, Phe |
| Tyr (Y) | Trp, Phe, Thr, Ser |
| Val (V) | Ile, Met, Leu, Phe, Ala, Norleucine |

In some embodiments, the BAFF polypeptide further comprises a heterologous amino acid sequence, e.g., a portion of one or more proteins other than BAFF, covalently bound to the BAFF portion at the latter's N- and/or C-terminus, and optionally further comprising a linker. The non-BAFF protein can be, for example, an immunoglobulin (e.g., the Fc portion of an immunoglobulin of any type or subtype (e.g., IgG (IgG₁, IgG₄), IgA, IgE, and IgM)), albumin, APRIL, or an affinity tag (e.g., myc-tag, His-tag, biotin, streptavidin, or GST). In one embodiment, BAFF is linked to a fluorescent protein, e.g., GFP or derivatives thereof. BAFF may also be linked to nonproteinaceous polymers, e.g., polyethylene glycol (PEG) and polypropylene glycol. In certain embodiments, BAFF is linked to a protein or other molecule that facilitates immobilization or detection of BAFF.

Additional BAFF compositions suitable for use in the methods of the invention and methods of making such compositions are described in, e.g., in U.S. Pat. Nos. 6,689,579; 6,475,986; 6,297,736; U.S. patent application Ser. No. 09/911,777; United States Patent Application Publication Nos. 2003/0175208; 2002/0064829; 2003/0022239; 2003/0095967; 2002/0037852; 2002/0055624; 2001/0010925; 2003/0023038; 2003/0119149; 2003/0211509; PCT Application Publication Nos. WO 99/117791; WO 00/43032; WO 98/27114; WO 98/18921; WO 98/55620; WO 99/12964; WO 99/11791; WO 00/39295; WO 00/26244; WO 01/96528; WO 02/15930; WO 03/033658; WO 03/022877; WO 03/040307; WO 03/050134; WO 03/035846; WO 03/060072; WO 03/060071; WO 04/016737; WO 00/43032; WO 00/47740; WO 00/45836; and EPC Application Publication No. 1146892.

The biological activity of a BAFF polypeptide may be evaluated using one or more of the following assays:
(1) B cell proliferation assay as described in, e.g., Scheider et al. (1999) J. Exp. Med., 189(11):1747-1756;
(2) B cell survival assay as described in, e.g., Batten et al. (2000) J. Exp. Med., 192(10):1453-1465;
(3) NFκB assay as described in, e.g., Claudio et al. (2002) Nature 1 mm., 3(10):898-899.
(4) Ig secretion assay as described in, e.g., Moore et al. (1999) Science, 285:260-263; and
(5) in vivo treatment of mice as described in, e.g., Moore et al. (1999) Science, 285:260-263.

The ability of BAFF to bind to one of its receptors (e.g., TACI, BCMA, BAFF-R) may optionally be used to pre-screen BAFF polypeptides before or in conjunction with evaluating their biological activity. Suitable receptor binding assays are described in, e.g., Gavin et al. (2003) J. Biol. Chem., 278(40):38220-38228. Accordingly, in some embodiments, the BAFF polypeptide comprises a fragment of the BAFF extracellular domain capable of binding to a BAFF receptor. It is contemplated that such a fragment may comprise one or more regions of at least 20, 30, 40, 50, 60, 70, 80, 90, 100, 110, or 120 contiguous amino acids of SEQ ID NO:1, SEQ ID NO:2, SEQ ID NO:3, or derivatives thereof.

The BAFF polypeptides of the invention include BAFF 60-mers and BAFF trimers. The structure of BAFF may be determined by assays described herein or as previously described in, e.g., Liu et al., Cell 108:383-394 (2002); Liu et al., Nature 423:49-56 (2003); Zhukovsky et al., Nature 427:413-414 (2004); and Hong et al., Nature 427:414 (2004). A BAFF 60-mer or a BAFF trimer of the invention may be present in a composition comprising other molecules, including other BAFF structures. For example, a BAFF 60-mer may be present in a composition comprising at least 10, 20, 30, 40, 50, 60, 70, 80, 85, 90, 95, or 99 percent 60-mer in relation to the total mass of BAFF polypeptides in the composition. Similarly, a BAFF trimer may be present in a composition comprising at least 10, 20, 30, 40, 50, 60, 70, 80, 85, 90, 95, or 99 percent trimer in relation to the total mass of BAFF polypeptides in the composition.

In one embodiment, the disclosure provides a BAFF trimer comprising a mutation in the DE loop, e.g., at least one substitution or deletion at His 218, Lys 216, or Glu223. For example, Lys 216 can be substituted with a natural or non-natural amino acid that has full or partial negative charge on any of its sidechain atoms as well as any organic moiety that has full or partial negative charge on any of its atoms; His 218 may be substituted with a natural or non-natural amino acid or organic moiety that has a molecular weight of 114 Da or lower; and/or Glu 223 may be substituted with a natural or non-natural amino acid that possesses full or partial positive charge on any of its sidechain atoms as well as any organic moiety that will have full or partial positive charge on any of its atoms.

In a further embodiment, the mutation is a deletion of at least one of Lys 216 and/or His 218; i.e., at least one monomeric subunit in the trimer comprises a deletion of Lys 216 and/or His 218. In one embodiment, the trimer includes at least one monomeric subunit having one of the following mutations: substitution of Lys 216 with aspartate (Asp) or glutamic acid (Glu); substitution of His 218 with glycine (Gly), alanine (Ala), or serine (Ser); and substitution of Glu 223 with arginine (Arg) or lysine (Lys). In further embodiments, the trimer includes at least one His218Ala mutation. In a further embodiment, at least one monomeric subunit in the trimer comprises a His218Ala mutation.

In one embodiment, the disclosure provides a BAFF 60-mer having the native sequence of BAFF in amino acids 217 to 224, or conservative substitutions thereof, and modified by at least one mutation in amino acids 134-216 or amino acids 225-285. In a further embodiment, the BAFF 60-mer includes at least one deletion in amino acids 134 to 145. In a further embodiment, amino acids 1 to 145 are deleted.

In one embodiment, the disclosure provides a BAFF 60-mer wherein at least one monomeric subunit comprises a substitution of His 218 with an amino acid selected from the group consisting of Trp, Phe, Tyr, Met, lie, and Leu.

### Methods of Making BAFF Polypeptides

The disclosure also provides methods of making a BAFF trimer or a BAFF 60-mer, applying techniques known in the art (see, e.g., Example 1 and Fernandez et al. (1999) Gene Expression Systems, Academic Press). In particular, the disclosure provides a method of making a BAFF trimer. The method involves preparing or constructing a BAFF polypeptide having at least one substitution or deletion at His 218, Lys 216 or Glu 223. In one embodiment, the BAFF polypeptide has at least one mutation selected from the following: substitution of Lys 216 with a natural or non-natural amino acid that has full or partial negative charge on any of its sidechain atoms or an organic moiety that has full or partial negative charge on any of its atoms; substitution of His 218 with a natural or non-natural amino acid or organic moiety that has a molecular weight of 114 Da or lower; and substitution of Glu 223 with a natural or non-natural amino acid that possesses full or partial positive charge on any of its sidechain atoms or any organic moiety that has full or partial positive charge on any of its atoms. For example, the method can include preparing or constructing a BAFF polypeptide having (or introducing in a BAFF polypeptide) at least one mutation selected from the following: substitution of Lys 216 with aspartate (Asp) or glutamic acid (Glu); substitution of His 218 with glycine (Gly), alanine (Ala), or serine (Ser); and substitution of Glu 223 with arginine (Arg) or lysine (Lys).

The disclosure also provides methods of making a BAFF 60-mer. In one embodiment, the disclosure provides a method of making a BAFF 60-mer, comprising constructing or preparing a BAFF polypeptide having a substitution of His 218 with a natural or non-natural amino acid or organic moiety that has a molecular weight of 115 Da or higher; or a substitution of Lys 216 and Glu 223 with a non-polar or uncharged aromatic natural or non-natural amino acid or organic moiety. In another embodiment, the disclosure provides a method of making a BAFF 60-mer, comprising introducing at least one mutation in amino acids 134-216 or amino acids 225-285 to a BAFF polypeptide having the native sequence, or conservative substitutions thereof, of amino acids 217 to 224. In a further embodiment, the BAFF 60-mer includes at least one deletion in amino acids 134 to 145. In a further embodiment, amino acids 1 to 145 are deleted. The disclosure also provides a method of making a BAFF 60-mer, comprising substituting His 218 with an amino acid selected from the group consisting of Trp, Phe, Tyr, Met, Ile, and Leu.

### Antibodies

The term “antibody,” as used herein, refers to immunoglobulin molecules and immunologically active portions of immunoglobulin molecules, i.e., molecules that contain an antigen binding site that specifically binds (immunoreacts with) an antigen, such as a BAFF polypeptide, including specific BAFF structures. The term antibody encompasses any polypeptide comprising an antigen-binding site of an immunoglobulin regardless of the source, species of origin, method of production, and characteristics. As a non-limiting example, the term “antibody” includes human, orangutan, monkey, mouse, rat, goat, sheep, and chicken antibodies. The term includes but is not limited to polyclonal, monoclonal, human, humanized, single-chain, chimeric, synthetic, recombinant, hybrid, mutated, resurfaced, and CDR-grafted antibodies. For the purposes of the present invention, it also includes, unless otherwise stated, antibody fragments such as Fab, F(ab′)₂, Fv, scFv, Fd, dAb, and other antibody fragments that retain the antigen-binding function. A “monoclonal antibody,” as used herein, refers to a population of antibody molecules that contain a particular antigen binding site and are capable of specifically binding to a particular epitope.

Antibodies can be made, for example, via traditional hybridoma techniques (Kohler et al., Nature 256:495-499 (1975)), recombinant DNA methods (U.S. Pat. No. 4,816,567), or phage display techniques using antibody libraries (Clackson et al., Nature 352:624-628 (1991); Marks et al., J. Mol. Biol. 222:581-597 (1991). For various other antibody production techniques, see Antibody Engineering, 2^{nd} ed., Borrebaeck, Ed., Oxford University Press, 1995; Antibodies: A Laboratory Manual, Harlow et al., Eds., Cold Spring Harbor Laboratory, 1988; and Antibody Engineering: Methods and Protocols (Methods in Molecular Biology), Lo, Ed., Humana Press, 2003An antibody may comprise a heterologous sequence such as an affinity tag, for example.

The term “antigen-binding domain” refers to the part of an antibody molecule that comprises the area specifically binding to or complementary to a part or all of an antigen. Where an antigen is large, an antibody may only bind to a particular part of the antigen. The “epitope” or “antigenic determinant” is a portion of an antigen molecule that is responsible for specific interactions with the antigen-binding domain of an antibody. An antigen-binding domain may be provided by one or more antibody variable domains (e.g., a so-called Fd antibody fragment consisting of a V_{H} domain). An antigen-binding domain comprises an antibody light chain variable region (V_{L}) and an antibody heavy chain variable region (V_{H}).

In one aspect, the disclosure provides isolated antibodies that bind one BAFF structure with a higher affinity than another BAFF structure, i.e., antibodies that preferentially bind a 60-mer or relative to a trimer, or vice versa. In some embodiments, the antibody preferentially binds BAFF 60-mer; in other embodiments, the antibody preferentially binds the BAFF trimer. In further embodiments, the binding constants for the antibody and the trimer and the antibody and the 60-mer, respectively, differ by at least a factor of 5, 10, 20, 30, 50, 100, 200, 500, 1000, or more. In part, the disclosure provides an antibody identified by any the methods described herein. The disclosure also provides pharmaceutical compositions comprising any of the aforementioned antibodies. In one embodiment, the pharmaceutical composition further comprises a suitable pharmaceutical excipient.

### “Isolated”

In some embodiments, the antibodies, polypeptides, or other compounds of the invention are isolated. The term “isolated” refers to a molecule that is substantially free of its natural environment. For instance, an isolated protein is substantially free of cellular material or other proteins from the cell or tissue source from which it was derived. The term also refers to preparations where the isolated protein is at least 70-80% (w/w) pure; or at least 80-90% (w/w) pure; or at least 90-95% pure; or at least 80%, 85%, 90%, 95%, 96%, 97%, 98%, 99%, or 100% (w/w) pure. In some embodiments, the isolated molecule is sufficiently pure for pharmaceutical compositions.

### Methods of Identifying or Evaluating Compounds

In one aspect, the disclosure provides a method of identifying a compound that binds one BAFF structure with a higher affinity than another BAFF structure, i.e., a compound that preferentially binds to a 60-mer relative to a trimer, or a compound that preferentially binds to a trimer relative to a 60-mer. In one aspect, the method includes the steps of providing a test compound, allowing the test compound to interact with a BAFF trimer and/or a 60-mer, determining whether the test compound preferentially binds the trimer or the 60-mer, and selecting a compound that binds one BAFF structure with a higher affinity than another BAFF structure. In some embodiments, the compound thus identified is an antibody, a peptide, an aptamer, or a small molecule. In certain embodiments, the BAFF trimer of the method includes a mutation of at least one amino acid in the DE loop. In a further embodiment, the trimer includes at least one monomeric subunit having one of the following mutations: substitution of Lys 216 with aspartate (Asp) or glutamate (Glu); substitution of His 218 with glycine (Gly), alanine (Ala), or serine (Ser); and substitution of Glu 223 with arginine (Arg) or lysine (Lys). In further embodiments, the trimer includes at least one His218Ala mutation.

In certain embodiments, the BAFF-binding compound is allowed to interact with a BAFF trimer and a BAFF 60-mer in separate but substantially identical experimental trials, and the results are compared to determine whether the compound preferentially binds the trimer or the 60-mer. In certain embodiments, a BAFF trimer and a BAFF 60-mer are allowed to compete for binding to the compound.

In one embodiment, whether a protein preferentially binds to a BAFF trimer or a BAFF 60-mer is determined using surface plasmon resonance, e.g., Biacore™, which is discussed in Examples 3 and 7. Additional exemplary binding assays include ELISA, protein or antibody microarrays, phage display, and assays routine in the art, including high throughput screening (HTS) methods.

The disclosure also provides a method of evaluating the activity of a BAFF-binding compound, comprising providing the compound, allowing it to interact with a BAFF trimer and/or a BAFF 60-mer, and determining the activity of the compound toward the BAFF trimer and the BAFF 60-mer (e.g., determining whether the compound preferentially binds, agonizes and/or inhibits BAFF trimer relative to BAFF 60-mer or vice versa. In some embodiments, the compound thus identified is an antibody, a peptide, an aptamer or a small molecule. In certain embodiments, the BAFF trimer utilized in the method includes a mutation of at least one amino acid in the DE loop. In a further embodiment, the trimer includes at least one monomeric subunit having one of the following mutations: substitution of Lys 216 with aspartate (Asp) or glutamate (Glu); substitution of His 218 with glycine (Gly), alanine (Ala), or serine (Ser); and substitution of Glu 223 with arginine (Arg) or lysine (Lys). In further embodiments, the trimer includes at least one His218Ala mutation.

The activity of a compound toward BAFF may be determined by treating BAFF with the compound and testing BAFF activity in one or more assays for BAFF activity. Exemplary assays of BAFF biological activity are known in the art, e.g.:
(1) B cell proliferation assay as described in, e.g., Scheider et al. (1999) J. Exp. Med., 189(11):1747-1756;
(2) B cell survival assay as described in, e.g., Batten et al. (2000) J. Exp. Med., 192(10):1453-1465;
(3) NFκB assay as described in, e.g., Claudio et al. (2002) Nature 1 mm., 3(10):898-899.
(4) Ig secretion assay as described in, e.g., Moore et al. (1999) Science, 285:260-263; and
(5) in vivo treatment of mice as described in, e.g., Moore et al. (1999) Science, 285:260-263.

The ability of BAFF to bind to one of its receptors (e.g., TACI, BCMA, BAFF-R) may be used in lieu of or in addition to the aforementioned assays of biological activity. Suitable receptor binding assays are described in, e.g., Gavin et al. (2003) J. Biol. Chem., 278(40):38220-38228.

In one embodiment, the compound thus evaluated is a BAFF antagonist, i.e., treatment of BAFF with the compound causes a measurable decrease in BAFF activity in one or more of the aforementioned assays. In an alternate embodiment, the compound evaluated is a BAFF agonist, i.e., treatment of BAFF with the compound causes a measurable increase in BAFF activity in one or more of the aforementioned assays.

In some embodiments, a method of the invention is used in conjunction with art-known methods for screening for compounds that bind BAFF in general. For example, a routine screening method is initially used to identify a BAFF-binding compound, which is then subjected to a method of the invention as a secondary screen, e.g., to evaluate the activity of the compound or to identify a compound that preferentially binds a 60-mer or a trimer.

### Binding Constants

Certain embodiments of the invention involve a consideration of binding constants. Exemplary binding constants include, but are not limited to, the equilibrium binding constant, K_{d}, and the kinetic binding constant, k_{d}. Techniques for determining binding constants are known in the art, e.g., surface plasmon resonance (Biacore™, discussed in Examples 3 and 7) and other methods described herein and elsewhere.

In one embodiment of the methods of the invention, a BAFF-binding compound is identified as preferentially binding either a BAFF trimer or a BAFF 60-mer if there is a difference in the binding constants for the interaction of the compound with the 60-mer and the compound with the trimer, respectively. In further embodiments, the compound is so identified if the difference in the binding constants is at least a factor of 5, 10, 20, 50, 100, 200, 500, 1000, or more.

In one embodiment of the invention, the binding constants for the interaction of the antibody with a BAFF trimer and the antibody with a BAFF 60-mer, respectively, differ by at least a factor of 5, 10, 20, 50, 100, 200, 500, 1000, or more.

### Computer Applications

The disclosure also provides computer-based methods and systems relating to the structures of BAFF. Exemplary embodiments include systems allowing the comparison of BAFF structures by displaying representations thereof on a computer screen; methods for determining the structure of a BAFF variant, derivative, fusion, or homologue; methods for designing a compound that preferentially binds, activates, and/or inhibits a BAFF trimer relative to a BAFF 60-mer, or vice versa; and methods for high throughput virtual screening for compounds that preferentially bind, activate, or inhibit a BAFF trimer relative to a BAFF 60-mer, or vice versa. For discussions of virtual screening methods, see Chin et al., Mini Rev. Med. Chem. 4:1053-1065 (2004) and Good, Curr. Opin. Drug Discov. Devel. 4:301-307 (2001). For a discussion of computer-based methods and systems with respect to the structure of BAFF, see WO 03/050134.

In some embodiments, the disclosure provides a machine readable storage medium which comprises structural data for BAFF trimer and BAFF 60-mer. Such storage medium encoded with these data are capable of displaying on a computer screen or similar viewing device, a three-dimensional graphical representation of BAFF trimer and BAFF 60-mer, which data and graphical representations can be used for comparison to (e.g., for virtual screening of) a database of compound structures for preferential binding to BAFF trimer or BAFF 60-mer. For example, a screening method can include docking a model of a test compound in a model of BAFF trimer and a model of BAFF 60-mer, and selecting the compound If it docks preferentially on the trimer or 60-mer.

### EXAMPLES

### Example 1

### Untagged BAFF Forms 60-mers in Solution

To test whether the formation of BAFF 60-mer is dependent on the presence of an N-terminal histidine tag as has been proposed (Zhukovsky et al., Nature 427:413-414 (2004)), a BAFF construct starting at amino acid Alanine 134 was engineered with no amino terminal tag. This construct is similar to that reported by Liu and coworkers, but lacking the histidine tag. Cell 108: 383-394 (2002).

Recombinant BAFF purified from Pichia pastoris is glycosylated at amino acid asparagine 242. Karpusas et al., J. Mol. Biol. 315: 1145-1154 (2002). It has been shown that soluble human BAFF expressed in 293T cells is not glycosylated. Schneider et al., J. Exp. Med. 189:1747-1756 (1999). Moreover, the construct reported by Liu and co-workers was purified from E. coli, and therefore it also was not glycosylated. Therefore, an additional construct was engineered with a mutation to glutamine at residue 242 (N242Q), to ensure that the yeast-expressed protein was not glycosylated. This protein is referred to as A134-BAFF-N242Q.

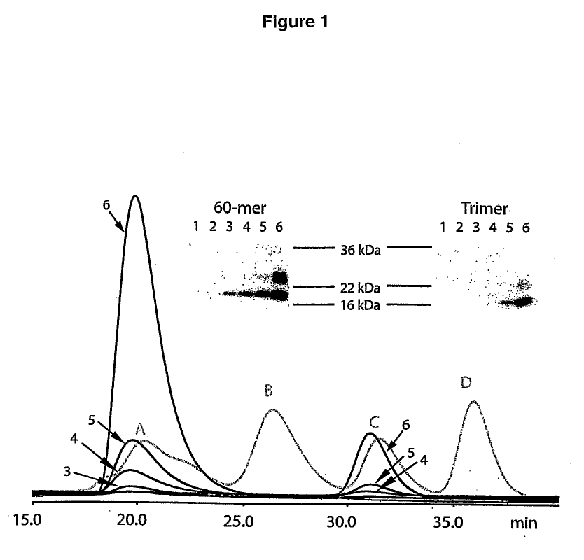

The oligomeric state of both A134-BAFF-N242Q and A134-BAFF in solution was evaluated by analytical gel filtration at pH 7.4. When 98 nM of Ala134-BAFF protein was loaded in a gel filtration column, the protein eluted as an oligomer (60-mer) (>670 kDa, FIG. 1) with a small portion eluting as a trimer (FIG. 1). To test if the formation of 60-mer is dependent on protein concentration, the elution profile of A134-BAFF was analyzed at different concentrations ranging from 98 nM to 245 pM of BAFF 60mer. As shown in FIG. 1A, A134-BAFF eluted as a 60-mer at all tested concentrations, suggesting that BAFF forms 60-mer at a concentration as low as 245 pM. In all cases, gel filtration was followed by Western blot analysis using an antibody against the carboxy terminus of BAFF and the results showed that the majority of BAFF was found in the fractions that eluted at the Mw expected for 60-mer (FIG. 1). Identical results were observed with Ala134-BAFF-N242Q. Similar results were obtained when the proteins were incubated in cell culture media (RPMI) for 24 or 48 hours (data not shown). Analytical gel filtration with in-line light scattering (GF-LS) at pH 7.2 was used to directly measure the molecular weight of the protein in solution. Wen et al., Anal. Biochemistry 240155-166 (1996). Table 2 shows that both A134-BAFF-N242Q and A134-BAFF proteins eluted with a molecular weight calculated from light scattering of 1,055 kDa. This value closely matches the molecular weight expected for BAFF 60-mer (1,022 kDa). In agreement with previous reports on histidine tagged BAFF (Liu et al., Cell 108:383-394 (2002); Zhukovsky et al., Nature 427:413-414 (2004)), a BAFF construct containing an N-terminal histidine tag (His-A134-BAFF) was also seen to exist exclusively as 60-mer by GF-LS (Table 2). The observation that both A134-BAFF and His-A134-BAFF are able to form oligomers in solution shows that BAFF 60-mer formation does not require an N-terminal histidine tag. In contrast, two constructs containing an N-terminal myc tag (myc-Q136-BAFF-N242Q and myc-Q136-BAFF) were shown to be exclusively trimeric by analytical gel filtration and GF-LS with a molecular weight of 54.60 and 55.75 kDa (Table 2).

TABLE 2

| | Molecular | Theoretical |
|---|---|---|
| | weight from | molecular weight |
| | Light-scattering | calculated |
| Proteins | (kDa) ± 5% | from sequence |
| A134-BAFF (60mer) | 1,055.00 | 1,022.40 |
| A134-BAFF-N242Q (60mer) | 959.00 | 1,023.24 |
| His-A134-BAFF (60mer) | 1,024.00 | 1,090.00 |
| Q136-BAFF (60mer) | 1,068.00 | 1,012.20 |
| Myc-Q136-BAFF (trimer) | 55.75 | 55.62 |
| Myc-Q136-BAFF-N242Q (trimer) | 54.60 | 55.66 |
| A134-BAFF-H218A (trimer) | 56.60 | 50.92 |

### Example 2

### The Formation of BAFF 60-mer is pH-Dependent and is Abolished by Mutating Residue Histidine 218 within the DE Loop

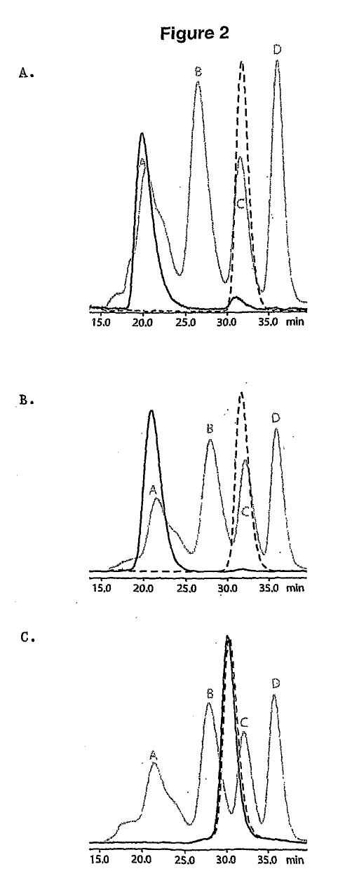

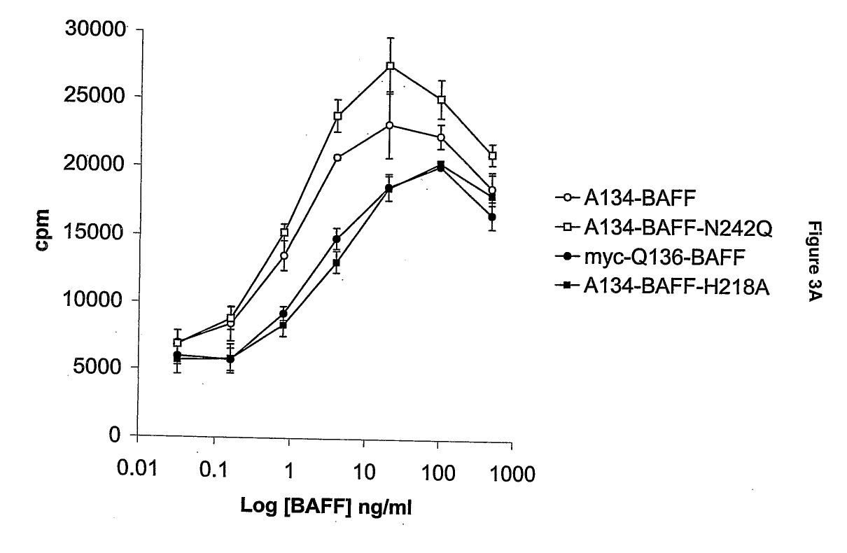

The initial study reporting the 60-mer formation of BAFF showed its pH dependency and suggested that the ionization state of two histidine residues in the DE loop might explain the pH dependency of the structure. Liu et al., Cell 108:383-394 (2002). A later report challenged this claim, suggesting rather that the pH dependency of the 60-mer formation was due to the ionization state of the histidine tag present in the construct used by Liu et al. Zhukovsky et al., Nature 427:413-414 (2004). The A134-BAFF and A134-BAFF-N242Q constructs, which lack an amino terminal histidine tag, provide a means to address this controversy. Accordingly, A134-BAFF-N242Q protein was dialyzed at different pHs from 5.0 to 8.0. The structural state was then determined by analytical gel filtration as shown in FIG. 2A. At pH 8.0, very little trimeric BAFF was detected; the protein eluted largely as a 60-mer with an apparent molecular weight greater than 670 kDa. However, at pH 5.0, BAFF eluted as a trimer (FIG. 2A). This result indicates that a form of purified BAFF that has no histidine tag at the amino terminus forms 60-mer in solution in a pH dependent manner.

To study the role of histidine 218 in 60-mer formation, histidine 218 was mutated to alanine, yielding A134-BAFF-H218A. The purified protein was characterized by analytical gel filtration at pH 7.5 (FIG. 2B) and also at pH 5.0 and pH 9.0 (data not shown). Unlike A134-BAFF, A134-BAFF-H218A was trimeric in solution (FIG. 2B), suggesting that the H218A mutation abolished 60-mer formation. This result extends the observation of Liu and coworkers (Cell 108:383-394 (2002)) that deletion of the entire DE loop disrupts 60-mer formation, by showing that the same result can be achieved by this single point mutation.

To determine whether myc-Q136-BAFF, which is exclusively trimeric at pH 7.5, can be induced to form 60-mers at high pH, the protein was dialyzed at pH 7.5 or pH 9.0 and evaluated by analytical gel filtration. As shown in FIG. 2C, myc-Q136-BAFF does not form 60-mers, even at high pH.

### Example 3

### BAFF Trimer and 60-mer have Distinct Levels of Activity

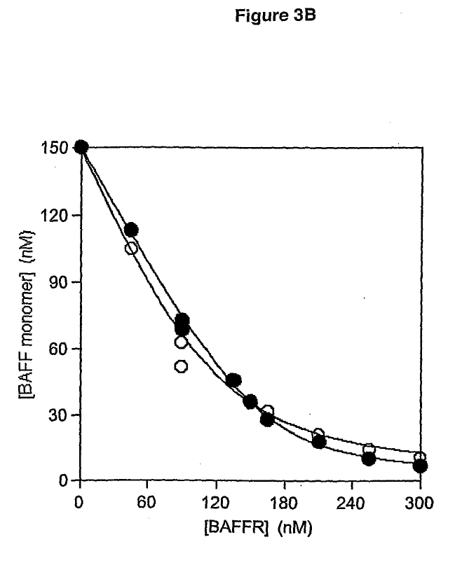

In the presence of IgM, BAFF co-stimulates the proliferation of B-cells. Schneider et al., J. Exp. Med. 189:1747-1756 (1999). FIG. 3A shows that oligomeric A134-BAFF is more efficacious than trimeric myc-BAFF in inducing B cell proliferation in vitro. Interestingly, the mutation H218A, which abolished 60-mer formation, resulted in activity in this assay that was identical to that of myc-Q136-BAFF (FIG. 3A). Thus myc-Q136-BAFF and A134-BAFF-H218A represent forms of BAFF that are unable to form 60-mer but retain biological activity. These results stand in contrast to those of Liu et al., who showed that preventing 60-mer formation by deleting the entire DE loop led to loss of activity in an NFκB assay. Liu et al., Cell 108:383-394 (2002). These results suggest that the fact that the DE loop is required for functional activity is not solely due to its role in mediating 60-mer formation.

To test whether the difference in functional activity observed between 60-mer and trimeric BAFF is due to a change in affinity for BAFFR, the affinity of BAFF trimer and 60-mer for soluble, monomeric BAFFR in solution was determined using Biacore (FIG. 3B). Titration of soluble, monomeric BAFFR against either 50 nM BAFF trimer (open circles) or 2.5 nM BAFF 60-mer (closed circles), equivalent to 150 nM BAFF monomers in each case, shows that BAFFR binds to both forms of BAFF with similar affinity (15 nM and 9 nM respectively), in agreement with previous results. Day et al., Biochemistry 44:1919-1931 (2005).

### Example 4

### Methods

### Expression and Purification of BAFF Polypeptides

A. BAFF Expression in Pichia pastoris

BAFF proteins were expressed in Pichia pastoris GS115 using the methanol inducible native AOX1 promoter or in the GS115 derivative MMC216 using the Doxycycline inducible TetO-AOX1 promoter. Expression plasmids used the alpha factor secretion signal and the HIS4 selectable marker. Manipulation and strain construction methods were as recommended by Invitrogen. StuI digestion was used to linearize DNA prior to transformation.

The N242Q and H218A mutations were constructed by QuikChange™ (Stratagene) site-directed mutagenesis. Proteins were expressed by shake flask induction in BMGY and BMMY (2% MeOH) according to Invitrogen recommendations for Myc-BAFF, or by fermentation in a reduced salts Basal Salts Hexametaphosphate medium for A134-BAFF-wt (A134-L285), A134-BAFF-H218A, His-A134-BAFF, A134-BAFF-N242Q and A134-BAFF-H218A-N242Q. Induction in fermenters was achieved with 1 μg/mL doxycyline or by MeOH fed-batch growth.

B. Purification of myc-Q136-BAFF (Q136-L285), A134-BAFF (A134-L285), A134-BAFF-H218A, A134-BAFF-N242Q, and His-A134-BAFF

The purification of myc-BAFF was as reported in Day et al., Biochemistry 44:1919-1931 (2005). Pichia pastoris supernatants expressing the different forms of BAFF were diafiltered by tangential flow filtration (Millipore™, Pellicon II™) and purified using ion exchange chromatography (Q XL Sepharose™, Q HP Sepharose™, and SP Sepharose™; Amersham Biosciences). Purified proteins were dialyzed against 10 mM Tris pH 7.5, 150 mM NaCl. His-A134-BAFF was purified on a nickel column. Proteins were over 90% pure as confirmed by SDS-PAGE and molecular mass determination was obtained by mass spectrometry.

### Gel Filtration

### A. Analytical Gel Filtration

BAFF proteins were dialyzed for 3 hours or overnight against the following buffers containing 150 mM NaCl: pH 5.0 (25 mM NaAcetate); pH 7.5 (10 mM Tris-HCl); pH 8.0 (20 mM Tris-HCl) and pH 9.0 (20 mM Tris-HCl). Proteins (0.5-1 mg) were then analyzed in a Superdex 200 10/30™ column (Amersham Biosciences) in the appropriate buffer.

B. Gel Filtration Assays with Light Scattering

Size exclusion chromatography (SEC) was carried out on a YMC-Pack Diol-120™ column 8.0×300 mm (YMC, Inc. Wilmington, N.C.) in 20 mM Na phosphate buffer pH 7.2, 150 mM NaCl (PBS) using a flow rate of 0.6 ml/min on a Waters Alliance™ instrument (Waters, Milford, Mass.). In addition to UV detection, the eluent was monitored in tandem with a refractive index detector (Waters, Milford, Mass.) and a Precision Detector PD2000™ light scattering instrument (Precision Detectors, Bellingham, Mass.). Static light scattering was measured on a Precision Detector PD2000/DLS™ instrument equipped with a dual angle flow cell detector. Molecular weight determination of each complex was performed with the Precision Detector™ Software.

### Solution Phase Affinity Measurements by Biacore

All experiments were performed on a Biacore 3000™ instrument (Biacore AB, Uppsala, Sweden). Chip preparation and solution phase binding measurements have been described in Day et al., Biochemistry 44:1919-1931 (2005). Briefly, monomeric BAFFR and the various forms of BAFF were mixed in different ratios in Biacore assay buffer (10 mM HEPES pH 7, 150 mM NaCl, 3.4 mM EDTA, 0.005% P-20 detergent, 0.1% BSA) and preincubated for a minimum of 3 hours at 4° C. to reach equilibrium. The equilibrated solutions were then injected over a BCMA-Fc derivitized surface and the concentration of free BAFF in solution was measured. The affinity of the interaction of BAFFR with various forms of BAFF was determined from a plot of the concentration of free BAFF binding to the BCMA-Fc derivitized chip versus receptor concentration by fitting the data to a quadratic binding equation as described in Day et al., Biochemistry 44:1919-1931 (2005).

### B Cell Proliferation Assay

The proliferation assay has been described in earlier reports. Thompson et al., Science 293:2108-2111 (2001) and Day et al., Biochemistry 44:1919-1931 (2005). Briefly, B cells isolated from mice splenocytes were incubated in the presence of 5 ug/ml of F(ab′)2 fragment goat anti-mouse IgM antibody and with different concentrations of different forms of BAFF for 48 h. Cells were pulsed for an additional 18 hours with [3H]-thymidine (1 uCi/well) and harvested. [3H]-Thymidine incorporation was monitored by liquid scintillation counting.

The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. The citation of any references herein is not an admission that such references are prior art to the present invention. To the extent that any general dictionary, technical dictionary, or material incorporated by reference contradicts or is inconsistent with this specification, the specification will supersede any such reference. The citation of any references herein is not an admission that such references are prior art to the present invention.

1. A method of identifying a BAFF-binding compound that preferentially binds a BAFF trimer over a BAFF 60-mer, or vice versa, the method comprising:
(a) providing a test compound;
(b) allowing the test compound to interact with a BAFF trimer and a BAFF 60-mer;
(c) determining whether the test compound preferentially binds the BAFF trimer or the BAFF 60-mer; and
(d) selecting the test compound If it preferentially binds the BAFF trimer over the BAFF 60-mer or vice versa,
thereby identifying said BAFF-binding compound.

2. The method of claim 1, wherein the test compound is allowed to interact with the BAFF trimer and the BAFF 60-mer in separate trials.

3. The method of claim 1, wherein the test compound is allowed to interact with the BAFF trimer and the BAFF 60-mer in a competition assay.

4. The method of claim 1, further comprising evaluating the selected compound for the ability to inhibit one or more of: BAFF-mediated B cell proliferation, BAFF-mediated B cell survival, and BAFF-mediated Ig secretion.

5. The method of claim 1, further comprising evaluating the selected compound for activity in an animal model of disease.

6. The method of claim 1, wherein the test compound is selected from the group consisting of an antibody, a peptide, an aptamer and a small molecule.

7. The method of claim 1, wherein the test compound is from a library selected from the group consisting of a phage display library, an aptamer library, and a small molecule library.

8. The method of claim 1, wherein at least one monomeric subunit in the BAFF trimer comprises at least one mutation selected from the following:
(a) substitution of Lys 216 with a natural or non-natural amino acid that has full or partial negative charge on any of its sidechain atoms or with an organic moiety that has full or partial negative charge on any of its atoms;
(b) substitution of His 218 with a natural or non-natural amino acid or organic moiety that has a molecular weight of 114 Da or lower; and
(c) substitution of Glu 223 with a natural or non-natural amino acid that possesses full or partial positive charge on any of its sidechain atoms or with any organic moiety that has full or partial positive charge on any of its atoms.

9. The method of claim 1, wherein at least one monomeric subunit in the BAFF trimer comprises at least one mutation selected from the following:
(a) a substitution of Lys 216 to an amino acid selected from the group consisting of Asp and Glu;
(b) a substitution of His 218 to an amino acid selected from the group consisting of Gly, Ala, and Ser; and
(c) a substitution of Glu 223 to an amino acid selected from the group consisting of Arg and Lys.

10. The method of claim 9, wherein at least one His 218 in the BAFF trimer is substituted with Ala.

11. An isolated antibody that preferentially binds either a BAFF trimer or a BAFF 60-mer with respect to each other.

12. The antibody of claim 11, wherein the antibody preferentially binds the BAFF trimer.

13. The antibody of claim 11, wherein the antibody preferentially binds the BAFF 60-mer.

14. The antibody of claim 11, wherein the binding constants for the antibody and a BAFF trimer and the antibody and a BAFF 60-mer differ by at least a factor of 10.

15. A pharmaceutical composition comprising the antibody of claim 11.

16. A trimeric BAFF polypeptide wherein at least one monomeric subunit in the trimer comprises at least one mutation selected from the following:
(a) a substitution of Lys 216 to an amino acid selected from the group consisting of Asp and Glu;
(b) a substitution of His 218 to an amino acid selected from the group consisting of Gly, Ala, and Ser; and
(c) a substitution of Glu 223 to an amino acid selected from the group consisting of Arg and Lys.

17. The trimeric BAFF polypeptide of claim 16 wherein at least one monomeric subunit comprises a His218Ala substitution.

18. A trimeric BAFF polypeptide wherein at least one monomeric subunit in the trimer comprises at least one mutation selected from the following:
(a) substitution of Lys 216 with a natural or non-natural amino acid that has full or partial negative charge on any of its sidechain atoms or with an organic moiety that has full or partial negative charge on any of its atoms;
(b) substitution of His 218 with a natural or non-natural amino acid or organic moiety that has a molecular weight of 114 Da or lower; and
(c) substitution of Glu 223 with a natural or non-natural amino acid that possesses full or partial positive charge on any of its sidechain atoms or with any organic moiety that has full or partial positive charge on any of its atoms.

19. A method of making a BAFF trimer, comprising introducing at least one mutation selected from the following:
(a) substitution of Lys 216 with a natural or non-natural amino acid that has full or partial negative charge on any of its sidechain atoms or with an organic moiety that has full or partial negative charge on any of its atoms;
(b) substitution of His 218 with a natural or non-natural amino acid or organic moiety that has a molecular weight of 114 Da or lower; and
(c) substitution of Glu 223 with a natural or non-natural amino acid that possesses full or partial positive charge on any of its sidechain atoms or with any organic moiety that has full or partial positive charge on any of its atoms.

20. A method of making a BAFF trimer, comprising introducing at least one mutation selected from the following:
(a) a substitution of Lys 216 to an amino acid selected from the group consisting of Asp and Glu;
(b) a substitution of His 218 to an amino acid selected from the group consisting of Gly, Ala, and Ser; and
(c) a substitution of Glu 223 to an amino acid selected from the group consisting of Arg and Lys.

21. A BAFF 60-mer, wherein at least one monomeric subunit in the 60-mer comprises:
(a) at least one mutation in amino acids 134 to 216 or amino acids 225 to 285; and
(b) the native sequence of BAFF from amino acid 217 to amino acid 224 or conservative substitutions thereof.

22. The BAFF 60-mer of claim 21, comprising at least one deletion in amino acids 134 to 145.

23. The BAFF 60-mer of claim 22, wherein amino acids 1 to 145 are deleted.

24. A BAFF 60-mer, wherein at least one monomeric subunit in the 60-mer comprises a substitution of His 218 with an amino acid selected from the group consisting of Trp, Phe, Tyr, Met, Ile, and Leu.

25. A method of making a BAFF 60-mer, comprising substituting His 218 with Trp, Phe, Tyr, Met, Ile, Leu or a natural or non-natural amino acid or organic moiety that has a molecular weight of 115 Da or higher.

26. A method of evaluating the activity of a BAFF-binding compound, comprising:
(a) providing a BAFF-binding compound;
(b) allowing the compound to interact with a BAFF trimer and/or a BAFF 60-mer;
(c) determining the activity of the compound toward the BAFF trimer and the BAFF 60-mer; and
(d) thereby evaluating the activity of the compound.

27. The method of claim 26, wherein at least one monomeric subunit in the BAFF trimer comprises at least one mutation selected from the following:
(a) a substitution of His 218 with a natural or non-natural amino acid or organic moiety that has a molecular weight of 115 Da or higher; and
(b) substitution of Lys 216 and Glu 223 with a non-polar or uncharged aromatic natural or non-natural amino acid or organic moiety.

28. The method of claim 26, wherein at least one monomeric subunit in the BAFF trimer comprises at least one mutation selected from the following:
(a) a substitution of Lys 216 to an amino acid selected from the group consisting of Asp and Glu;
(b) a substitution of His 218 to an amino acid selected from the group consisting of Gly, Ala, and Ser; and
(c) a substitution of Glu 223 to an amino acid selected from the group consisting of Arg and Lys.


SEQUENCE LISTING (3 sequences)

SEQ ID NO: 1 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 2 - protein, 309 aa, Mus musculus
MDESAKTLPPPCLCFCSEKGEDMKVGYDPITPQKEEGAWFGICRDGRLLAATLLLALLSSSFTAMSLYQLAALQADLMNLRMELQSYRGSATPAAAGAPELTAGVKLLTPAAPRPHNSSRGHRNRRAFQGPEETEQDVDLSAPPAPCLPGCRHSQHDDNGMNLRNIIQDCLQLIADSDTPTIRKGTYTFVPWLLSFKRGNALEEKENKIVVRQTGYFFIYSQVLYTDPIFAMGHVIQRKKVHVFGDELSLVTLFRCIQNMPKTLPNNSCYSAGIARLEEGDEIQLAIPRENAQISRNGDDTFFGALKLL

SEQ ID NO: 3 - protein, 288 aa, Gallus gallus
MKSVDCVHVIQQKDTASSPSGPPGAASGTTGLFSVTFLWLAMLLSSCLAAVSLYHAITLKTELEALRSELIYRVRARSPLEQPPVSPGDKKAGASVSSFLQVSAAGARQENRLPGPSPAESFQTEIWDRNRNRGRRSIVNAEETVLQACLQLIADSKSDIQQKDDSSIVPWLLSFKRGTALEEQGNKIVIKETGYFFIYGQVLYTDTTFAMGHLIQRKKAHVFGDDLSLVTLFRCIQNMPQSYPNNSCYTAGIAKLEEGDELQLTIPRRRAKISLDGDGTFFGAVRLL